SEQUENCE LISTING (21 sequences)

SEQ ID NO: 1 - protein, 8 aa, Synthetic construct
PHYKHRLH

SEQ ID NO: 2 - protein, 8 aa, Synthetic construct
  SITE 1: note = D-Proline
  SITE 2: note = D-Histidine
  SITE 3: note = D-Tyrosine
  SITE 4: note = D-Lysine
  SITE 5: note = D-Histidine
  SITE 6: note = D-Arginine
  SITE 7: note = D-Leucine
  SITE 8: note = D-Histidine
PHYKHRLH

SEQ ID NO: 3 - protein, 12 aa, Synthetic construct
PHYKHRLHGPPR

SEQ ID NO: 4 - protein, 12 aa, Synthetic construct
  SITE 1: note = D-Proline
  SITE 2: note = D-Histidine
  SITE 3: note = D-Tyrosine
  SITE 4: note = D-Lysine
  SITE 5: note = D-Histidine
  SITE 6: note = D-Arginine
  SITE 7: note = D-Leucine
  SITE 8: note = D-Histidine
  SITE 9: note = D-Glycine
  SITE 10: note = D-Proline
  SITE 11: note = D-Proline
  SITE 12: note = D-Arginine
PHYKHRLHGPPR

SEQ ID NO: 5 - protein, 17 aa, Synthetic construct
  SITE 1: note = D-Threonine
  SITE 2: note = D-Proline
  SITE 15: note = D-Threonine
  SITE 16: note = D-Proline
  SITE 17: note = D-Threonine
TPPHYKHRLHGPPRTPT

SEQ ID NO: 6 - protein, 13 aa, Synthetic construct
GRKKRRQRRRPPQ

SEQ ID NO: 7 - protein, 13 aa, Synthetic construct
  SITE 1: note = D-Glycine
  SITE 2: note = D-Arginine
  SITE 3: note = D-Lysine
  SITE 4: note = D-Lysine
  SITE 5: note = D-Arginine
  SITE 6: note = D-Arginine
  SITE 7: note = D-Glutamine
  SITE 8: note = D-Arginine
  SITE 9: note = D-Arginine
  SITE 10: note = D-Arginine
  SITE 11: note = D-Proline
  SITE 12: note = D-Proline
  SITE 13: note = D-Glutamine
GRKKRRQRRRPPQ

SEQ ID NO: 8 - protein, 25 aa, Synthetic construct
PHYKHRLHGPPRGRKKRRQRRRPPQ

SEQ ID NO: 9 - protein, 25 aa, Synthetic construct
  SITE 13: note = D-Glycine
  SITE 14: note = D-Arginine
  SITE 15: note = D-Lysine
  SITE 16: note = D-Lysine
  SITE 17: note = D-Arginine
  SITE 18: note = D-Arginine
  SITE 19: note = D-Glutamine
  SITE 20: note = D-Arginine
  SITE 21: note = D-Arginine
  SITE 22: note = D-Arginine
  SITE 23: note = D-Proline
  SITE 24: note = D-Proline
  SITE 25: note = D-Glutamine
PHYKHRLHGPPRGRKKRRQRRRPPQ

SEQ ID NO: 10 - protein, 30 aa, Synthetic construct
TPPHYKHRLHGPPRGRKKRRQRRRPPQTPT

SEQ ID NO: 11 - protein, 30 aa, Synthetic construct; identical to SEQ ID NO: 10
  SITE 1: note = D-Threonine
  SITE 2: note = D-Proline
  SITE 28: note = D-Threonine
  SITE 29: note = D-Proline
  SITE 30: note = D-Threonine

SEQ ID NO: 12 - protein, 5 aa, Synthetic construct
PHYKH

SEQ ID NO: 13 - protein, 8 aa, Synthetic construct
GGHPHYKH

SEQ ID NO: 14 - protein, 6 aa, Synthetic construct
GGHRLH

SEQ ID NO: 15 - protein, 8 aa, Synthetic construct
PHYKHGGH

SEQ ID NO: 16 - protein, 6 aa, Synthetic construct
RLHGGH

SEQ ID NO: 17 - protein, 8 aa, Synthetic construct
  SITE 1: note = D-Glycine
  SITE 2: note = D-Glycine
  SITE 3: note = D-Histidine
  SITE 4: note = D-Proline
  SITE 5: note = D-Histidine
  SITE 6: note = D-Tyrosine
  SITE 7: note = D-Lysine
  SITE 8: note = D-Histidine
GGHPHYKH

SEQ ID NO: 18 - protein, 8 aa, Synthetic construct
  SITE 4: note = D-Proline
  SITE 5: note = D-Histidine
  SITE 6: note = D-Tyrosine
  SITE 7: note = D-Lysine
  SITE 8: note = D-Histidine
GGHPHYKH

SEQ ID NO: 19 - protein, 21 aa, Synthetic construct
GGHPHYKHGRKKRRQRRRPPQ

SEQ ID NO: 20 - protein, 26 aa, Synthetic construct
TPGGHPHYKHGRKKRRQRRRPPQTPT

SEQ ID NO: 21 - protein, 8 aa, Synthetic construct
RLHPHYKH

(19) United States
(12) Patent Application Publication
(10) Pub. No.: US 2024/0287477 A1
(43) Pub. Date: Aug. 29, 2024

(54) CATALASE MIMETIC PEPTIDES

(71) Applicant: Northwestern University, Evanston, IL (US)

(72) Inventors: Marcelo BONINI, Evanston, IL (US); Flavio Romero PALMA, Evanston, IL (US); Fernando Toshio OGATA, Evanston, IL (US)

(21) Appl. No.: 18/583,685

(22) Filed: Feb. 21, 2024

Related U.S. Application Data
(60) Provisional application No. 63/486,207, filed on Feb. 21, 2023.
(60) Provisional application No. 63/490,463, filed on Mar. 15, 2023.

(51) Int. Cl. C12N 9/08; A61K 38/00; A61P 35/00

(52) U.S. Cl. CPC C12N 9/0065; A61P 35/00; C12Y 111/01006; A61K 38/00

(57) ABSTRACT

Provided herein are peptides that mimic the activity of endogenous catalase. In particular, catalase mimetic peptides and methods of use thereof for the treatment or prevention of diseases such as cancer are provided.

### CROSS-REFERENCE TO RELATED APPLICATION

The present invention claims priority to U.S. Provisional Application No. 63/486,207, filed Feb. 21, 2023, and U.S. Provisional Application No. 63/490,463, filed Mar. 15, 2023, which are herein incorporated by reference in their entirety.

### SEQUENCE LISTING

The text of the computer readable sequence listing filed herewith titled “NWEST_41681_203_SequenceListing.xml,” created May 13, 2024, having a file size of 44,332 bytes, is hereby incorporated by reference in its entirety.

### FIELD

Provided herein are peptides that mimic the activity of endogenous catalase. In particular, catalase mimetic peptides and methods of use thereof for the treatment or prevention of diseases such as cancer are provided.

### BACKGROUND

Transcription stability enforces cellular identity and is tightly controlled by restrictions imposed on both transcription factor function and target gene accessibility. Progression of cancer to metastasis and multi-drug resistance requires fluid transcriptional programs that can explore different genomic landscapes to enable clonal expansion of aggressive and treatment resistant phenotypes. Treatments that enforce restrictions on transcription factor function and target gene accessibility are needed to prevent cancer development, progression, metastasis, and the formation of drug resistance.

### SUMMARY

Provided herein are peptides that mimic the activity of endogenous catalase. In particular, catalase mimetic peptides and methods of use thereof for the treatment or prevention of diseases such as cancer are provided.

Certain embodiments provided herein are summarized below. Additional aspects are described in the Detailed Description of the Invention, Examples, Drawings, and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention.

In some embodiments, provided herein are compositions comprising a peptide capable of binding two copper ions and having a redox potential of less than 2.0 V, wherein the peptide comprises at least one amino acid substitution, amino acid addition, or amino acid steroisomerization relative to SEQ ID NO: 1. In some embodiments, the peptide exhibits enhanced resistance to digestion (e.g., proteinase digestion) in a biological environment compared to a peptide of SEQ ID NO: 1. In some embodiments, the peptide is cell permeable. In some embodiments, the peptide is capable of nuclear localization. In some embodiments, the peptide comprises 0-4 amino acid substitutions (e.g., 0, 1, 2, 3, 4, or ranges therebetween) relative to SEQ ID NO: 1. In some embodiments, the peptide comprises one or more D-amino acids. In some embodiments, the peptide comprises 0-4 amino acid substitutions (e.g., 0, 1, 2, 3, 4, or ranges therebetween) relative to SEQ ID NO: 1, and one or more D-amino acids. In some embodiments, the peptide comprises a C-terminal and/or N-terminal cap sequence, for example, to reduce digestion (e.g., proteinase digestion) in a biological environment. In some embodiments, the C-terminal cap is TPT and/or the N-terminal cap is TP, or stereoisomeric variants thereof. In some embodiments, the peptide comprises a cell penetrating peptide (CPP) sequence. In some embodiments, the CPP has at least 70% (e.g., 70%, 75%, 80%, 85%, 90%, 95%, or more) sequence identity with SEQ ID NO: 6 or 7. In some embodiments, the peptide comprises two copper-binding motifs selected from PHYKH, RLH, and GGH, or stereoisomeric variants thereof. In some embodiments, the peptide comprises at least 70% (e.g., 70%, 75%, 80%, 85%, 90%, 95%, or more) sequence identity with one of SEQ ID NOS: 1-5 and 8-20, or stereoisomeric variants thereof.

In some embodiments, provided herein are polynucleotides encoding a peptide described herein. In some embodiments, provided herein are vectors comprising the polynucleotides described herein. In some embodiments, provided herein are pharmaceutical formulations comprising peptides, polynucleotides, and/or vectors described herein, and a pharmaceutical carrier. In some embodiments, provided herein are methods of treating or preventing a disease comprising administering to a subject a pharmaceutical formulation described herein (e.g., comprising a peptide, polynucleotide, or vector herein). In some embodiments, the disease is a cancer and the administering reduces the severity, therapeutic resistance, or metastatic potential of the cancer. In some embodiments, methods further comprise co-administering a chemotherapeutic, immunotherapeutic, or other cancer therapy. In some embodiments, the pharmaceutical formulation comprises a peptide and administering comprises contacting the cells of the subject with the peptide under conditions such that the peptide enters the cells. In some embodiments, the pharmaceutical formulation comprises a polynucleotide or vector and administering comprises contacting the cells of the subject with the polynucleotide or vector under conditions such that the peptide encoded by the polynucleotide or vector is expressed by the cells and/or is incorporated into a genome of the cells. In some embodiments, provided herein are methods comprising introducing a nucleic acid sequence encoding a peptide described herein into the genome of a cell or subject (e.g., by a CRISPR-based system).

### BRIEF DESCRIPTION OF THE DRAWINGS

FIG. 1A. REDOX effects promoted by peptidyl di-copper complex. (A) Oxidized (λex=405 nM) and reduced (λex=488 nM) roGFP2 signals 30 minutes after exposure to the metallocomplex (Day 1) and at the final of the experiment (Day 5) were measured and the oxidized/reduced ratio was obtained using ImageJ (shown as histogram). FIG. 1B. Representative images of MCF-10AER/cSrc transformation after treatment with 4-hydroxy-tamoxifen (1 μM) registered by Lionheart (phase contrast). FIG. 1C. Western blot analysis of E-Cadherin (E-Cad), Fibronectin (FN1), and Vimentin (Vim) in MCF-10AER/cSrc transformed cells (5 days after 4-OHT treatment). CATm1-peptidyl di-copper complex, as previously reported, DNK01-peptidyl di-copper complex with D-amino acids, sd-single dose, cd-daily doses.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the phrase “symptoms are reduced” means one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

As used herein, the term “patient” typically refers to a subject that is being treated for a disease or condition.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

As used herein, the term “preventing” refers to prophylactic steps taken to reduce the likelihood of a subject (e.g., an at-risk subject) developing or suffering from a particular disease, disorder, or condition (e.g., asthma). The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the term “treatment” (also “treat” or “treating”) refer to obtaining a desired pharmacologic and/or physiologic effect against a particular disease, disorder, or condition. Preferably, the effect is therapeutic, i.e., the effect partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the terms “administration” and “administering” refer to the act of to introducing a substance, such as a drug, prodrug, or other agent, or therapeutic treatment to a subject or in vivo, in vitro, or ex vivo cells, tissues, and organs. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. Exemplary routes of administration to the human body can be by parenteral administration (e.g., intravenously, subcutaneously, etc.), orally, etc.

As used herein, the term “approximately” and “about” is intended to encompass normal statistical variation as would be understood by those of ordinary skill in the art as appropriate to the relevant context. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would 15 exceed 100% of a possible value).

As used herein, the term “human” is a human is an embryo, a fetus, an infant, a child, a teenager, an adult, or a senior citizen.

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

The term “amino acid” refers to natural amino acids, unnatural amino acids, and amino acid analogs, all in their D and L stereoisomers, unless otherwise indicated, if their structures allow such stereoisomeric forms. Embodiments herein refer to various amino acid abbreviations (single-letter or three-letter abbreviations) that will be understood by those in the field. Any amino acid abbreviations not defined herein refer to their field-accepted meaning. For example, “NMe” preceding an amino acid name refers to an “N-methyl” group on the amino acid, “Nle” is “norleucine,” “Abu” is “α-Aminobutyric acid,” “Aib” is “2-Aminoisobutyric acid,” “Nal(2′) is “3-(2-Naphthyl)-L-alanine,” “tic” is “1,2,3,4-tetrahydroisoquinoline-3-carboxylic acid,” “HpH” is “homophenylalanine,” “Bip” is “N-alpha-Fmoc-beta-(4-biphenyl)-L-alanine,” “D-Phe(4tBu)” is “D-4-tert-butyl-phenylalanine,” and the single-letter or three-letter abbreviations for the common proteinogenic amino acids are provided below.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyroolysine may also be considered proteinogenic amino acids.

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “substitution” refers to replacement of one amino acid for a different amino acid within a sequence of amino acids. Amino acids are stereoisomers, existing as L- or D-amino acids. The term “isomeric substitution” refers to replacing the D-isomer for the L-isomer of an amino acid within a sequence. Unless specified as an “isomeric substitution,” amino acid substitutions herein involve chemical changes, not merely changes from L- to D-isomer (“non-isomeric substitutions”).

The term “peptide” refers an oligomer to short polymer of amino acids linked together by peptide bonds. In contrast to other amino acid polymers (e.g., proteins, polypeptides, etc.), peptides are of about 30 amino acids or less in length. Peptides herein may be of 5 amino acids, 10 amino acids, 15 amino acids, 20 amino acids, 25 amino acids, 30 amino acids, or any integer values or ranges therebetween. A peptide may comprise natural amino acids, non-natural amino acids, proteinogenic amino acids, non-proteinogenic amino acids, amino acid analogs, and/or modified amino acids. A peptide may be a subsequence of naturally occurring protein or a non-natural (artificial) sequence.

The term “conservative”, in reference to an amino acid substitution, refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid I;
3) Asparagine (N) and Glutamine (Q);
4) Arginine I and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine I and Methionine (M).
   Naturally occurring residues may be divided into classes based on common side chain properties, for example: polar positive (or basic) (histidine (H), lysine (K), and arginine I); polar negative (or acidic) (aspartic acid (D), glutamic acid I); polar neutral (serine (S), threonine (T), asparagine (N), glutamine (Q)); non-polar aliphatic (alanine (A), valine (V), leucine (L), 25 isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

The term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.); (2) determining the number of positions containing identical (or similar) monomers (e.g., same amino acids occurs in both sequences, similar amino acid occurs in both sequences) to yield the number of matched positions; (3) dividing the number of matched positions by the total number of positions in the comparison window (e.g., the length of the longer sequence, the length of the shorter sequence, a specified window); and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 20 amino acids) may have up to X substitutions (e.g., 2) relative to SEQ ID NO:Z, and may therefore also be expressed as “having X (e.g., 2) or fewer substitutions relative to SEQ ID NO:Z.”

The term “vector” refers to a macromolecule or complex of molecules comprising a polynucleotide or protein to be delivered to a host cell, either in vitro or in vivo. A vector can be a modified RNA, a lipid nanoparticle (encapsulating either DNA or RNA), a transposon, an adeno-associated virus (AAV) vector, an adenovirus, a retrovirus, an integrating lentiviral vector (LVV), or a non-integrating LVV. Thus, as used herein “vectors” include naked polynucleotides used for transformation (e.g., plasmids) as well as any other composition used to deliver a polynucleotide to a cell, included vectors capable of transducing cells and vectors useful for transfection of cells. “Vector systems” refers to combinations of one, two, three, or more vectors used to delivery one, two, three, or more polynucleotides.

The term “genetic modification” refers to a permanent or transient genetic change induced in a cell following introduction of new nucleic acid (i.e., nucleic acid exogenous to the cell). Genetic modification can be accomplished by incorporation of the new nucleic acid into the genome of the cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, CRISPR based technology (e.g., CRISPR-cas9 systems), and the like.

### DETAILED DESCRIPTION

Provided herein are peptides that mimic the activity of endogenous catalase. In particular, catalase mimetic peptides and methods of use thereof for the treatment or prevention of diseases such as cancer are provided.

Nuclear reactive oxygen species (ROS) have recently been shown to drive epigenetic modifications with consequent epithelial-mesenchymal transition (EMT)-induced phenotype (Palma et al.; incorporated by reference in its entirety) Using the MCF-10A-derivative cell line model ((Hirsch, Iliopoulos, Tsichlis, & Struhl, 2009) nuclear ROS levels of untreated control cells were compared with 4-hydroxytamoxifen-differentiated MCF-10A^{ER/cSrc}, conditions which have shown a significant difference in nuclear ROS levels, with more ROS detected in the later. This difference was also observed in other breast cancer cell lines (i.e., MDA-MB-231, BT20, among others). These experiments identified nuclear ROS as an important driver of such alterations, cells expressing nuclear catalase, an enzyme that converts H₂O₂ (a ROS molecule) into H₂O, were unable to acquire some of the EMT phenotype, assessed by specific protein markers (SOX9 and Fibronectin). The expression profile in both conditions changed due to a chromatin remodeling event promoted by H3.1 oxidation in specific genomic locations and later substituted by H3.3 protein. It has been demonstrated that H3.1 is related to heterochromatin and H3.3 to euchromatin, a closed and open states of chromatin, respectively.

Experiments were conducted during development of embodiments herein to develop a druggable antioxidant mimetic with the ability to interfere with MCF-10A^{ER/cSrc} transformation and other tumors malignant phenotypes.

Catalase (CAT) is a metalloenzyme (EC 1.11.1.6) with a MW of approximately 60 kDa comprising 527 amino acid residues. It converts H₂O₂ into H₂O and O₂. A short peptidyl di-copper complex has been developed via a combinatorial approach to mimic CAT (CATm1; SEQ ID NO: 1; PHYKHRLH) (Koudedja Coulibaly, 2021; incorporated by reference in its entirety). Provided herein are modified versions of the CATm1di-copper bidding peptide.

In some embodiments, provided herein are CATm1 variants comprising one or more D amino acids. In some embodiments, one or more (e.g., 1, 2, 3, 4, 5, 6, 7, 8) of P1, H2, Y3, K4, H5, R6, L7, and/or H8 are D-amino acids (e.g., D-versions of the L-amino acids present in CATm1). In some embodiments, a peptide is provided herein in which all of the positions of unmodified CATm1 are D-amino acids (dPdHdYdKdHdRdLdH; SEQ ID NO: 2 (aka, DNK01)). In some embodiments, peptides comprising the amino acid sequence of CATm1 but with any combination of one or more (e.g., 1, 2, 3, 4, 5, 6, 7, 8) D-amino acids, such as SEQ ID NO: 1, SEQ ID NO: 2, or any intermediate combinations thereof.

In some embodiments, provided herein are peptides comprising one or more (e.g., 1, 2, 3, 4) amino acid substitutions (i.e., non-isomeric substitutions). In some embodiments, a peptide herein comprises one or more (e.g., 1, 2, 3, 4) non-isomeric (and/or isomeric) substitutions relative to SEQ ID NO: 1 and/or 2. In some embodiments, a peptide comprises 1-4 conservative substitutions relative to SEQ ID NO: 1 and/or 2. In some embodiments, a peptide comprises 1-4 semi-conservative substitutions relative to SEQ ID NO: 1 and/or 2. In some embodiments, a peptide comprises 1-4 non-conservative substitutions relative to SEQ ID NO: 1 and/or 2. Substituted amino acids may be L- or D-amino acids. In some embodiments, substitutions may be for non-proteinogenic amino acids, amino acid analogs, etc.

In some embodiments, a peptide herein comprises additional amino acids (e.g., 1, 2, 3, 4, 5, 6, 7, 8, 9, 10, 11, 12, 13, 14, 15, 16, 17, 18, 19, 20, 22, 24, 26, 28, 30, or more) fused to the N- and/or C-terminus of a peptide sequence herein (e.g., SEQ ID NO: 1-2 or isomeric variants thereof). In some embodiments, additional amino acids may be L- or D-amino acids. Additional amino acids may be for any suitable purpose, such as, but not limited to solubility, cell permeability, protection from digestion (e.g., proteinase digestion), biocompatibility, localization (e.g., cellular localization, nuclear localization), target engagement, co-factor (e.g., Cu2+) binding, activity, etc. In some embodiments, a peptide herein comprises a GPPR tetramer (or isomeric variants thereof (e.g., dGdPdPdR)) fused to the C-terminus, N-terminus, or at an internal cite. For example, in some embodiments, a peptide comprising PHYKHRLHGPPR (SEQ IF NO: 3) is provided. In some embodiments, any combination of the amino acids of such a peptide may be D- or L-amino acids, for example:
PHYKHRLHGPPR (SEQ ID NO: 3)
dPdHdYdKdHdRdLdHdGdPdPdR (SEQ ID NO: 4; aka, DNK01);
and any other combinations of D- and L-amino acids.
   In some embodiments, a GPPR or dGdPdPdR tetramer or other modifying peptide group is fused to a peptide having one or more substitutions relative to a peptide herein (e.g., SEQ ID NO: 1, 2, or an isomeric variant thereof).

In some embodiments, a peptide herein comprises a C-terminal and/or N-terminal cap sequence, for example, to reduce degradation of the peptide within a cellular or biological environment. For example, any peptide herein may comprise an N-terminal TP dipeptide (e.g., TP, dTdP. dTP, or TdP). In some embodiments, a peptide herein comprises a C-terminal TPT tripeptide (e.g., TPT, TPdT, TdPT, TdPdT, dTPT, dTPdT, dTdPT, or dTdPdT,). For example, a peptide herein may comprise dTdPPHYKHRLHGPPRdTdPdT (SEQ ID NO: 5; aka, DNK02).

As addressed above, any of the peptides herein may be fused to an additional peptide sequence, for example, to provide various functionalities of characteristics to the peptides herein. In some embodiments, a cell penetrating peptide is fused to a peptide sequence herein (internally, terminally, between the peptide sequence and a cap sequence, etc.) to promote cell entry of the peptides herein. Any cell penetrating peptide sequences understood in the filed may find use in embodiments herein. In some embodiments, the peptide herein is fused to a TAT motif (e.g., GRKKRRQRRRPPQ (SEQ ID NO: 6), dGdRdKdKdRdRdQdRdRdRdPdPdQ (SEQ ID NO: 7), or other isomeric variants thereof). For example, in some embodiments, a peptide of PHYKHRLHGPPRGRKKRRQRRRPPQ (SEQ ID NO: 8), or isomeric variants thereof, PHYKHRLHGPPRdGdRdKdKdRdRdQdRdRdRdPdPdQ (SEQ ID NO: 9), or isomeric variants thereof, or TPPHYKHRLHGPPRGRKKRRQRRRPPQTPT (SEQ ID NO: 10), or isomeric variants thereof (e.g., dTdPPHYKHRLHGPPRGRKKRRQRRRPPQdTdPdT (SEQ ID NO: 11; aka DNK03)), are provided. Other exemplary cell-penetrating peptides that may find use in embodiments herein (e.g., fused to the peptides herein) include Antennapedia sequences, Penetratin, Antp-3A (Antp mutant), Buforin II, Transportan, MAP (model amphipathic peptide), K-FGF, Ku70, Prion, pVEC, Pep-1, SynB1, Pep-7, I-IN-1, BGSC (Bis-Guanidinium-Spermidine-Cholesterol, and BGTC (Bis-Guanidinium-Tren-Cholesterol).

The PHYKHRLH sequence (SEQ ID NO: 1) comprises two copper-binding motifs (PHYKH (SEQ ID NO: 12) and RLH). In some embodiments, a peptide herein comprises one or both of the PHYKH and RLH copper-binding motifs, but in a distinct arrangement. For example, a peptide may comprise PHYKH only, RLH only, or PHYKH and RLH separated by a linker or other peptide, RLHPHYKH (SEQ ID NO. 21), etc. In some embodiments, a peptide may comprise one of PHYKH or RLH with an different copper-binding motif (e.g., GGH). Exemplary GGH-containing peptides herein may comprise (GGHPHYKH (SEQ ID NO: 13), GGHRLH (SEQ ID NO: 14), PHYKHGGH (SEQ ID NO: 15), RLHGGH (SEQ ID NO: 16), or any isomcric variants thereof (e.g., dGdGdHdPdHdYdKdH (SEQ ID NO: 17), GGHdPdHdYdKdH (SEQ ID NO: 18), etc.). In some embodiments, any peptide with a suitable combination of two copper-binding motifs may find use in the peptides herein. The aforementioned two—copper-binding-motif peptides may be further substituted (e.g., 1-4 non-isomeric substitutions), isomerized (any combination of D- and L-amino acids) fused (e.g., to any of the peptide modifiers (e.g., TAT motif) described herein), etc. For example, a peptide may comprise GGHPHYKHGRKKRRQRRRPPQ (SEQ ID NO: 19), TPGGHPHYKHGRKKRRQRRRPPQTPT (SEQ ID NO: 20), isomeric variants thereof, or other variants or combinations of the elements and substitutions described herein.

Provided herein are methods for the treatment or prevention of disease (or reduction in symptoms thereof) which results from oxidative damage to cells, proteins, and/or DNA in a subject. More particularly, provided herein are methods for using a regulatory mechanism to control chromatin states that enable cancer progression and drug resistance acquisition.

The method is not limited by the nature of the target cells, proteins, and/or DNA. In some embodiments, the target is a cell. More specifically, in some embodiments, the cells are stem cells, cancer cells, cancer stem cells, nerve cells, blood cells, muscle cells, skin cells, nerve cells, endothelial cells, or pancreatic cells. Samples for use in the methods herein include, but are not limited to, tissue sections, blood, saliva, cerebral spinal fluid, pleural fluid, milk, lymph, sputum and semen.

The methods are not limited by the nature of the condition which results from the oxidative damage. In some embodiments, the condition being treated is cancer. More specifically, in some embodiments, the condition being treated is anal cancer, bladder cancer, bone cancer, breast cancer, cervical cancer, childhood cancer, colon cancer, hereditary cancer, kidney cancer, leukemia, lung cancer, lymphoma, oral cancer, pancreatic cancer, prostate cancer, skin cancer, or throat cancer. In some embodiments, the condition being treated is inflammation. In some embodiments, the condition being treated is free radical damage to the skin. In some embodiments, the condition being treated is aging. In some embodiments, the condition being treated is diabetes. In some embodiments, the condition being treated is neurodegenerative disease.

The methods herein are not limited by the subject. In some embodiments, the subject being treated is an animal. More specifically, in some embodiments, the subject being treated a cat, dog, cow, pig, chicken, or other non-human animal. In some embodiments, the subject being treated is a human. More specifically, in some embodiments, the subject being treated is a male or female.

In certain embodiments, peptides herein are combined with one or more additional agents to form pharmaceutical compositions. Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a peptide disclosed herein or salts thereof with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of peptide described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The peptide disclosed herein, can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include a peptide described herein, can be formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

One may administer the peptides in a local rather than systemic manner, for example, via injection of the compound directly into an organ or tissue, often in a depot preparation or sustained release formulation. Such long-acting formulations may be administered by implantation (for example subcutaneously or intramuscularly) or by intramuscular injection. Furthermore, one may administer the drug in a targeted drug delivery system, for example, in a liposome coated with organ specific antibody. The liposomes will be targeted to and taken up selectively by the organ. In addition, the drug may be provided in the form of a rapid release formulation, in the form of an extended-release formulation, or in the form of an intermediate release formulation.

Generally, a peptide herein is administered in an amount effective for amelioration of, or prevention of the development of symptoms of, the disease or disorder (i.e., a therapeutically effective amount). Thus, a therapeutically effective amount can be an amount that is capable of at least partially preventing or reversing a disease or disorder. The dose required to obtain an effective amount may vary depending on the agent, formulation, disease or disorder, and individual to whom the agent is administered. Determination of effective amounts may also involve in vitro assays in which varying doses of agent are administered to cells in culture and the concentration of agent effective for ameliorating some or all symptoms is determined in order to calculate the concentration required in vivo. Effective amounts may also be based in in vivo animal studies.

A peptide herein can be administered prior to, concurrently with and subsequent to the appearance of symptoms of a disease or disorder. In some embodiments, an agent is administered to a subject with a family history of the disease or disorder, or who has a phenotype that may indicate a predisposition to a disease or disorder, or who has a genotype which predisposes the subject to the disease or disorder.

In some embodiments, the compositions described herein are provided as pharmaceutical and/or therapeutic compositions. The pharmaceutical and/or therapeutic compositions of the present invention can be administered in a number of ways depending upon whether local or systemic treatment is desired and upon the area to be treated. Administration can be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Compositions and formulations for topical administration can include transdermal patches, ointments, lotions, creams, gels, drops, suppositories, sprays, liquids and powders.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi dose containers, with an added preservative.

Dosing and administration regimes are tailored by the clinician, or others skilled in the pharmacological arts, based upon well-known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, the compounds are administered to a subject at a dose of about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone. Dosing may be once per day or multiple times per day for one or more consecutive days.

In therapeutic applications, the compositions are administered to a patient already suffering from a disease, in an amount sufficient to cure or at least partially arrest the symptoms of the disease. Amounts effective for this use will depend on the severity and course of the disease, previous therapy, the patient's health status, weight, and response to the drugs, and the judgment of the treating clinician.

In prophylactic applications, compositions containing the peptides herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating clinician.

The peptides described herein also may be used in combination with procedures that may provide additional or synergistic benefit to the patient.

When the peptides and pharmaceutical compositions herein are used for treating cancer, they may be co-administered with one or more chemotherapeutics. Many chemotherapeutics are presently known in the art and can be used in combination with the peptides herein. In some embodiments, the chemotherapeutic is selected from the group consisting of mitotic inhibitors, alkylating agents, anti-metabolites, intercalating antibiotics, growth factor inhibitors, cell cycle inhibitors, enzyme inhibitors, topoisomerase inhibitors, protein-protein interaction inhibitors, biological response modifiers, anti-hormones, angiogenesis inhibitors, and anti-androgens. The peptides and pharmaceutical compositions herein may also be co-administered with various immunotherapeutic (e.g., monoclonal antibodies, immune checkpoint inhibitors, bispecific engagers, CAR-T cells, etc.) that are understood in the field. In some embodiments, the peptides and pharmaceutical compositions herein are co-administered with surgery, radiation, or other non-drug-related treatments.

In some embodiments, provided herein are polynucleotides encoding the peptides described herein. In some embodiments, the polynucleotide is introduced into a cell and/or subject under conditions to allow for expression of the encoded peptide. In some embodiments, the polynucleotide is within an expression vector that allows for introduction of the polynucleotide into the subject/cells and expression of the peptide form the polynucleotide.

In some embodiments, a polynucleotide encoding a peptide herein is delivered to cells by a viral or non-viral vector. In some embodiments, the peptide herein is encoded in a DNA or RNA polynucleotide, optionally delivered by any viral or non-viral method known in the art. In some embodiments, the disclosure provides methods comprising contacting cells with a lipid nanoparticle comprising a DNA or mRNA encoding the peptide herein. In some embodiments, the methods of the disclosure comprise contacting cells with a virus comprising a DNA or RNA (e.g., a DNA genome, a negative-sense RNA genome, a positive-sense RNA genome, or a double-stranded RNA genome) encoding the peptide herein. In some embodiments, the virus is selected from a retrovirus, adenovirus, AAV, non-integrating lentiviral vector (LVV), and an integrating LVV. In some embodiments, the cells are transfected with a plasmid. In some embodiments, the plasmid comprises a polynucleotide encoding the peptide herein. In some embodiments, the plasmid comprises a transposon comprising the peptide herein.

In some embodiments, the polynucleotides encoding the peptide herein may be codon-optimized or otherwise altered so long as the functional activity of the encoded gene is preserved. In some embodiments, the polynucleotides encode a modified or variant of a peptide herein, including truncations, insertions, deletions, or fragments, so long as the functional activity of the peptide herein is preserved.

In some embodiments, a nucleic acid encoding peptide herein is operably linked to a promoter and/or enhancer to facilitate expression of the peptide. Depending on the host/vector system utilized, any of a number of suitable transcription and translation control elements, including constitutive and inducible promoters, transcription enhancer elements, transcription terminators, etc. may be used in the expression vector (e.g., Bitter et al. (1987) Methods in Enzymology, 153:516-544). Non-limiting examples of suitable eukaryotic promoters (promoters functional in a eukaryotic cell) include CMV, CMV immediate early, HSV thymidine kinase, early and late SV40, long terminal repeats (LTRs) from retrovirus, and mouse metallothionein-I. In some embodiments, promoters that are capable of conferring neuronal-specific expression will be used.

Various techniques may be employed for introducing nucleic acid molecules and/or vectors encoding the peptides herein into cells. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. Other examples include: N-TER Nanoparticle Transfection System by Sigma-Aldrich, FECTOFLY transfection reagents for insect cells by Polyplus Transfection, Polyethylenimine “Max” by Polysciences, Inc., Unique, Non-Viral Transfection Tool by Cosmo Bio Co., Ltd., LIPOFECTAMINE LTX Transfection Reagent by Invitrogen, SATISFECTION Transfection Reagent by Stratagene, LIPOFECTAMINE Transfection Reagent by Invitrogen, FUGENE HD Transfection Reagent by Roche Applied Science, GMP compliant IN VIVO-JETPEI transfection reagent by Polyplus Transfection, and Insect GENEJUICE Transfection Reagent by Novagen.

Techniques in the field of recombinant genetics can be used to facilitate introduction into and expression from a polynucleotide or vector. Basic texts disclosing general methods of recombinant genetics include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994).

Suitable viral vectors for use in embodiments herein include, but are not limited to, viral vectors (e.g. viral vectors based on vaccinia virus; poliovirus; adenovirus (e.g., Li et al. (1994) Invest Opthalmol Vis Sci 35:2543-2549; Borras et al. (1999) Gene Ther 6:515-524; Li and Davidson, (1995) Proc. Natl. Acad. Sci. 92:7700-7704; Sakamoto et al. (1999) Hum Gene Ther 5: 1088-1097; WO 94/12649; WO 93/03769; WO 93/19191; WO 94/28938; WO 95/11984 and WO 95/00655); adeno-associated virus (e.g., Ali et al. (1998) Hum Gene Ther 9(1):81-86, 1998, Flannery et al. (1997) Proc. Natl. Acad. Sci. 94:6916-6921; Bennett et al. (1997) Invest Opthalmol Vis Sci 38:2857-2863; Jomary et al. (1997) Gene Ther 4:683-690; Rolling et al. (1999), Hum Gene Ther 10:641-648; Ali et al. (1996) Hum Mol Genet. 5:591-594; WO 93/09239, Samulski et al. (1989) J. Vir. 63:3822-3828; Mendelson et al. (1988) Virol. 166: 154-165; and Flotte et al. (1993) Proc. Natl. Acad. Sci. 90: 10613-10617; SV40; herpes simplex virus; human immunodeficiency virus (e.g., Miyoshi et al. (1997) Proc. Natl. Acad. Sci. 94: 10319-10323; Takahashi et al. (1999) J Virol 73:7812-7816); a retroviral vector (e.g., Murine-Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available. The following vectors are provided by way of example; for eukaryotic cells: pXT1, pSG5 (Stratagene), pSVK3, pBPV, pMSG, pSVLSV40 (Pharmacia), and pAd (Life Technologies). However, any other suitable vector may be used.

In some embodiments, route of administration and pharmaceutical formulation are selected to provide efficient and effective delivery of the peptides, polynucleotides, or vectors described herein. In some embodiments, a peptide or polynucleotide is provided with a suitable carrier. In some embodiments, a peptide or polynucleotide is encapsulated or embedded into a carrier. In some embodiments, a carrier may comprise a liposome, nanoparticle, or other suitable system for delivery of peptides or polynucleotides. In some embodiments, a peptide or polynucleotide is conjugated to a carrier molecule. Suitable carrier molecules for conjugation may include small molecules, peptides, proteins, polymers, etc. In some embodiments, the carrier and/or delivery system is selected to optimize the solubility, stability, bioavailability, targeting, etc. of the peptide or polynucleotide.

In certain embodiments, a carrier for a peptide or polynucleotide herein comprises lipid particles, for example, as described in Kanasty R, Nat Mater. 12(11):967-77 (2013), which is hereby incorporated by reference. In some embodiments, the lipid-based carrier is a lipid nanoparticle, which is a lipid particle between about 1 and about 500 nanometers in size. In some embodiments, the lipid-based vector is a lipid or liposome. Liposomes are artificial spherical vesicles comprising a lipid bilayer. In some embodiments, the lipid-based vector is a small nucleic acid-lipid particle (SNALP). SNALPs comprise small (less than 200 nm in diameter) lipid-based nanoparticles that encapsulate a nucleic acid. In some embodiments, the SNALP is useful for delivery of polynucleotide (e.g., minigene).

In some embodiments, peptides or polynucleotides are delivered via polymeric carriers. In some embodiments, the polymeric carrier is a polymer or polymerosome. Polymers encompass any long repeating chain of monomers and include, for example, linear polymers, branched polymers, dendrimers, and polysaccharides. Linear polymers comprise a single line of monomers, whereas branched polymers include side chains of monomers. Dendrimers are also branched molecules, which are arranged symmetrically around the core of the molecule. Polysaccharides are polymeric carbohydrate molecules, and are made up of long monosaccharide units linked together. Polymersomes are artificial vesicles made up of synthetic amphiphilic copolymers that form a vesicle membrane, and may have a hollow or aqueous core within the vesicle membrane. Various polymer-based systems can be adapted as a vehicle for administering DNA or RNA encoding the peptides herein. Exemplary polymeric materials include poly(D,L-lactic acid-co-glycolic acid) (PLGA), poly(caprolactone) (PCL), ethylene vinyl acetate polymer (EVA), poly(lactic acid) (PLA), poly(L-lactic acid) (PLLA), poly(glycolic acid) (PGA), poly(L-lactic acid-co-glycolic acid) (PLLGA), poly(D,L-lactide) (PDLA), poly(L-lactide) (PLLA), PLGA-b-poly(ethylene glycol)-PLGA (PLGA-bPEG-PLGA), PLLA-bPEG-PLLA, PLGA-PEG-maleimide (PLGA-PEG-mal), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (polyacrylic acids), and copolymers and mixtures thereof, polydioxanone and its copolymers, polyhydroxyalkanoates, polypropylene fumarate), polyoxymethylene, poloxamers, poly(ortho)esters, poly(butyric acid), poly(valeric acid), poly(lactide-co-caprolactone), trimethylene carbonate, polyvinylpyrrolidone, polyorthoesters, polyphosphazenes, Poly([beta]-amino esters (PBAE), and polyphosphoesters, and blends and/or block copolymers of two or more such polymers. Polymer-based systems may also include Cyclodextrin polymer (CDP)-based nanoparticles such as, for example, CDP-admantane (AD)-PEG conjugates and CDP-AD-PEG-transferrin conjugates. Exemplary polymeric particle systems for delivery of drugs, including nucleic acids, include those described in U.S. Pat. Nos. 5,543,158, 6,007,845, 6,254,890, 6,998,115, 7,727,969, 7,427,394, 8,323,698, 8,071,082, 8,105,652, US 2008/0268063, US 2009/0298710, US 2010/0303723, US 2011/0027172, US 2011/0065807, US 2012/0156135, US 2014/0093575, WO 2013/090861, each of which are hereby incorporated by reference in its entirety.

In some embodiments, a nucleic acid encoding a peptide berein is inserted into the genetic material of a host using a CRISPT-based system, for example, a CRISPR/Cas9 system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). CRISPRs are DNA loci comprising short repetitions of base sequences. Each repetition is followed by short segments of “spacer DNA” from previous exposures to a virus. CRISPRs are often associated with Cas genes that code for proteins related to CRISPRs. The CRISPR/Cas system is a prokaryotic immunc system that confers resistance to foreign genetic elements such as plasmids and phages and provides a form of acquired immunity. CRISPR spacers recognize and cut these exogenous genetic elements in a manner analogous to RNAi in eukaryotic organisms. The CRISPR/Cas system may be used for gene editing. By delivering the Cas (e.g., Cas9) protein and appropriate guide RNAs into a cell, the organism's genome can be cut at any desired location. Methods for using CRISPR/Cas9 systems, and other systems, for insertion of a gene (e.g., encoding a peptide herein) into a host cell to produce an engineered cell are described in, for example, U.S. Pub. No. 20180049412; herein incorporated by reference in its entirety. One or more elements of a CRISPR system that may find use in embodiments herein can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for a target sequence and fixed tracrRNA) are introduced into the cell. In general, target sites at the 5′ end of the gRNA target the Cas nuclease to the target site, causing complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., encoding a peptide herein). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site, followed by disruptions or alterations as discussed herein. Paired nickases can be used, e.g., to improve specificity, each directed by a pair of different gRNAs targeting sequences such that upon introduction of the nicks simultaneously, a 5′ overhang is introduced. In some embodiments, the CRISPR system is used to incorporate a polynucleotide encoding a peptide herein into the genome of a cell or subject in a manner that allows for expression of the peptide by the host cellular expression machinery.

### EXPERIMENTAL

### Peptide Synthesis

Peptides were synthesized by standard fluorenylmethoxycarbonyl (Fmoc) solid-phase peptide synthesis using a CEM model Liberty Blue Microwave Assisted Peptide Synthesizer on rink amide MBHA resin. Automated coupling reactions were performed using 4 equiv. Fmoc-protected amino acid, 4 equiv. of N,N′-diisopropylcarbodiimide (DIC), and 8 equiv. ethyl(hydroxyimino)cyanoacetate (Oxyma pure). Removal of the Fmoc groups was achieved with 20%4-methylpiperidine in DMF. Peptides were cleaved from the resin using standard solutions of 95% TFA, 2.5% water, 2.5% triisopropylsilane (TIS) and precipitated with cold ether.”

### Peptide Conjugation to Copper (II)

Lyophilized peptides were resuspended in MOPS buffer (30 mM, pH 7.5) to a concentration of 5 mM; a 50 mM copper solution was prepared in the same buffer. Both solutions were mixed in a 1:2 (peptide:copper) concentration 30 minutes before applying them to the cell culture. The final concentration of the complex in the cell culture was peptide at 250 μM and CuSO4 at 500 μM. The culture was monitored by measuring the ratio I(405/525)/I(488/525) given by the NLS-roGFP2-Orp1.

### Results

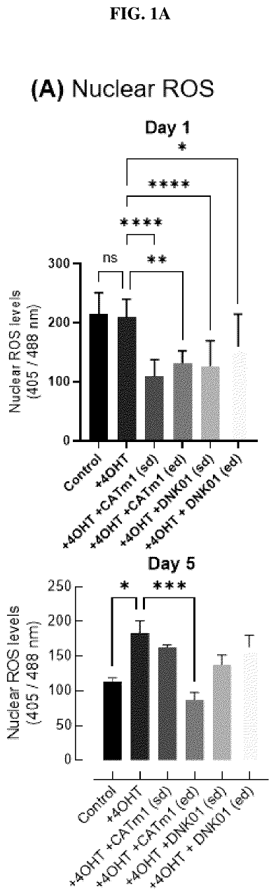

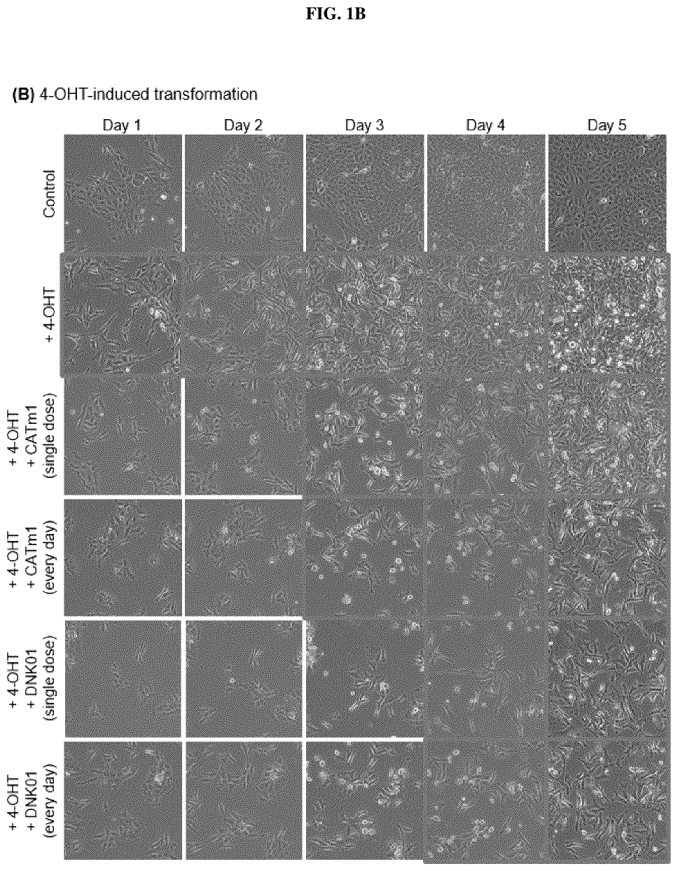

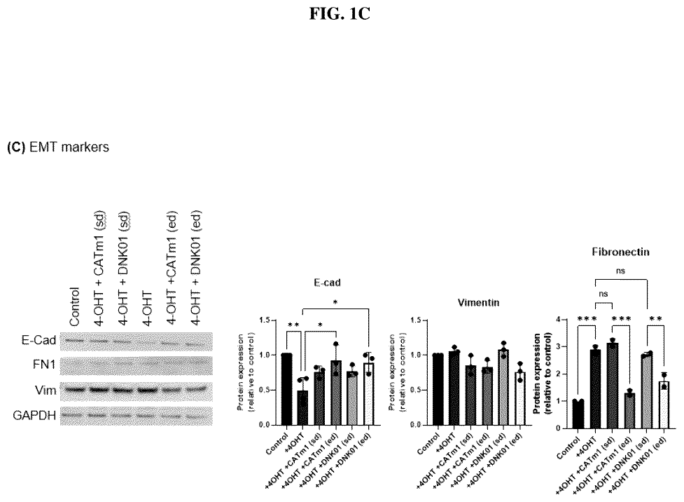

Exposure to 4-hydroxytamoxifen (4-OHT) transforms MCF-10A ER/cSrc/NLS-Orp1-roGFP2 cells into a tumorous cell line due to the oxidation of the nuclear compartment. Experiments were conducted by measuring the nuclear ROS in these cells. Probe oxidation status was monitored for 5 days after exposure to 4-OHT (FIG. 1A). As observed, CATm1, in its L-amino acids form, reduced the nuclear environment, presenting lower levels of nuclear oxidants right after exposure (FIG. 1A—day 1—top panel). If Cu²⁺-CATm1 was presented only as single dose in day 1, the complex would disrupt at some point and the ROS levels increase (FIG. 1A—day 5—lower panel). Whereas, presenting the cells with Cu²⁺-CATm1 everyday with fresh media was important to keep the ROS in lower levels (FIG. 1A). It was not sufficient to keep the ROS levels as low as on day 1. This was due to a disruption of the complex and accumulation of free copper in the media.

Another important observation was the delayed transformation of the cells in the system. After exposure to 4-OHT, cells acquired a spiked shape as soon as day 1 to day 2 (FIG. 1B). This process was delayed for two days when the cells were exposed to Cu²⁺-CATm1 or Cu²⁺-DNK01 either in single dose or every day. This indicates that ROS are driving the transformation of these cells after 4-OHT exposure.

Experiments were conducted during development of embodiments herein to measure the expression of EMT markers in this system at the end of the experiment (day 5). As shown in FIG. 1C, the epithelial marker E-cadherin has a higher expression in the control cells and a tendency to keep the expression in cells exposed both to 4-OHT and the peptides in both conformations. But in cells exposed only to 4-OHT, the levels dropped to almost 50% in comparison to the control cells. In the same FIGURE, the mesenchymal markers Vimentin and Fibronectin were also probed by western blot. Although Vimentin did not have a profound change in its expression levels, Fibronectin had an interesting behavior. Transformed cells, 4-OHT treated, expressed 3 times compared to control, and cells also treated with peptides in single doses. But cells treated with peptides every day during the experiment failed to over express this marker.

### REFERENCES

The following references are herein incorporated by reference in their entireties.
Flavio R. Palma, F. T. (2022). H3.1Cys96 oxidation by mitochondrial ROS promotes chromatin remodeling, breast cancer progression to metastasis and multi-drug resistance. bioRxiv.
Gisela Tünnemann, R. M. (2006). Cargo-dependent mode of uptake and bioavailability of TAT-containing proteins and peptides in living cells. The FASEB Journal, 1775-1784.
Hirsch, H. A., Iliopoulos, D., Tsichlis, P. N., & Struhl, K. (2009). Metformin Selectively Targets Cancer Stem Cells, and Acts Together with Chemotherapy to Block Tumor Growth and Prolong Remission. Cancer Res, 7507-7511.
Koudedja Coulibaly, M. T. (2021). A di-Copper Peptidyl Complex Mimics the Activity of Catalase, a Key Antioxidant Metalloenzyme. Inorg. Chem., 9309-9319.
Mark Nitz Dr., K. J. (2003). A Powerful Combinatorial Screen to Identify High-Affinity Terbium(III)-Binding Peptides. ChemBioChem, 272-276.
Regina Tugyi, K. U. (2005). Partial D-amino acid substitution: Improved enzymatic stability and preserved Ab recognition of a MUC2 epitope peptide. Proc Natl Acad Sci US A., 413-418.
Sarkar, C. H. (1997). Amino Terminal Cu(II)- and Ni(II)—Binding (ATCUN) Motif of Proteins and Peptides: Metal Binding, DNA Cleavage, and Other Properties. Acc. Chem. Res., 123-130.

1. A composition comprising a peptide capable of binding two copper ions and having a redox potential of less than 2.0 V, wherein the peptide comprises at least one amino acid substitution, amino acid addition, or amino acid steroisomerization relative to SEQ ID NO: 1.

2. The composition of claim 1, wherein the peptide exhibits enhanced resistance to digestion in a biological environment compared to a peptide of SEQ ID NO: 1.

3. The composition of claim 1, wherein the peptide is cell permeable.

4. The composition of claim 1, wherein the peptide is capable of nuclear localization.

5. The composition of claim 1, wherein the peptide comprises 0-4 amino acid substitutions relative to SEQ ID NO: 1, and wherein the peptide comprises one or more D-amino acids.

6. The composition of claim 1, wherein the peptide comprises a C-terminal and/or N-terminal cap sequence to reduce digestion in a biological environment.

7. The composition of claim 6, wherein the C-terminal cap is TPT and the N-terminal cap is TP, or stereoisomeric variants thereof.

8. The composition of claim 1, wherein the peptide comprises a cell penetrating peptide (CPP) sequence.

9. The composition of claim 8, wherein the CPP has at least 70% sequence identity with SEQ ID NO: 6 or 7.

10. The composition of claim 1, wherein the peptide comprises two copper-binding motifs selected from PHYKH, RLH, and GGH, or stereoisomeric variants thereof.

11. The composition of claim 1, wherein the peptide comprises at least 70% sequence identity with one of SEQ ID NOS: 1-5 and 8-20, or stereoisomeric variants thereof.

12. A polynucleotide encoding the peptide of the composition of claim 1.

13. The polynucleotide of claim 12, wherein the polynucleotide is DNA or RNA.

14. (canceled)

15. A vector comprising the polynucleotide of claim 12.

16. (canceled)

17. A method of treating or preventing a disease comprising administering to a subject the pharmaceutical formulation of claim 1.

18. The method of claim 17, wherein the disease is cancer and the administering reduces the severity, therapeutic resistance, or metastatic potential of the cancer.

19. The method of claim 17, further comprising co-administering a chemotherapeutic, immunotherapeutic, or other cancer therapy.

20. The method of claim 17, wherein the pharmaceutical formulation comprises a peptide and administering comprises contacting the cells of the subject with the peptide under conditions such that the peptide enters the cells.

21. The method of claim 17, wherein the pharmaceutical formulation comprises a polynucleotide or vector and administering comprises contacting the cells of the subject with the polynucleotide or vector under conditions such that the peptide encoded by the polynucleotide or vector is expressed by the cells and/or is incorporated into a genome of the cells.

22. A method comprising introducing a nucleic acid sequence encoding the peptide of the composition of claim 1 into the genome of a cell or subject.
